(19) United States
(12) Patent Application Publication
(10) Pub. No.: US 2005/0180932 A1
(43) Pub. Date: Aug. 18, 2005

(54) Ultraviolet absorption compositions

(75) Inventor: Tatsuya Hattori, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC, Chuo-ku (JP)

(21) Appl. No.: 11/024,794

(22) Filed: Dec. 30, 2004

Related U.S. Application Data
(63) Continuation of application No. PCT/JP03/08380, filed on Jul. 1, 2003.

(30) Foreign Application Priority Data
Jul. 4, 2002 (JP) 2002-196242

(51) Int. Cl. A61K 7/42

(52) U.S. Cl. 424/59

(57) ABSTRACT

Ultraviolet absorption materials comprising an amide-containing guanidine derivative of the following general formula (I): 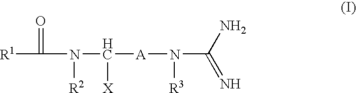 or a salt thereof are disclosed. The materials have high solubility, and can be blended as ultraviolet absorption materials with a variety of cosmetics and are adsorbed well by skin and/or hair.

### CROSS REFERENCES TO RELATED APPLICATIONS

This application is a continuation of International Patent Application No. PCT/JP03/08380, filed on Jul. 1, 2003, and claims priority to Japanese Patent Application No. 196242/2002, filed on Jul. 4, 2002, both of which are incorporated herein by reference in their entireties.

### BACKGROUND OF THE INVENTION

1. Field of the Invention

The present invention relates to novel water-soluble ultraviolet (UV) absorption compositions containing an amide-containing guanidine derivative or its salt. The present invention also relates to methods for protecting skin and hair by topically applying such a composition to the skin or hair. 2. Discussion of the Background

So far, as methods for protecting skin or hair from ultraviolet ray, a UV scattering method using a metal oxide such as zinc oxide or titanium oxide and a method for absorbing UV with an organic compound such as a cinnamic acid-type or benzophenone-type compound have been employed.

Such metal oxides, however, are not soluble in water or other solvents and further are not adsorbed by the skin or hair, and accordingly, their use has been limited to uses such as foundation cosmetics.

The problem with the organic UV absorbents is that most of them are oil soluble and, thus, require the addition of an emulsifying agent or the like when blended with aqueous cosmetics, which gives an unfavorable feeling on use. There is another problem in that the organic UV absorbent is washed away with sweat, which decreases the expected effect of UV absorption, since it imparts no adsorption capability to the skin or hair similarly to the metal oxide.

Therefore, it has been desired to develop a water-soluble UV absorbent which is readily adsorbed by the skin or hair and which resists rinsing-out.

On the other hand, the compounds having a guanidine group have been utilized in a wide variety of fields such as drugs, agrochemicals, bactericides, insecticides, valuable metal scavengers, chelating agents, and the like; in particular, the amide-containing guanidine derivatives or their salts are known to be used as surfactants as described in JP-A-2-243614/1990. It has not yet been known that a guanidine derivative or salt thereof to which an UV-absorbing group is attached through an amide linkage can be utilized as an UV absorbent.

Thus, there remains a need for UV-absorbing compositions which are free of the above-drawbacks. There also remains a need for methods of protecting skin and hair from UV radiation by topically applying such a composition.

### SUMMARY OF THE INVENTION

Accordingly, it is one object of the present invention to provide novel UV-absorbing materials.

It is another object of the present invention to provide novel water-soluble UV-absorbing materials.

It is another object of the present invention to provide novel water-soluble UV absorbing materials which are readily adsorbed by hair and/or skin and are excellent in resistance to rinsing-out.

It is another object of the present invention to provide novel UV absorption compositions which contain such a UV-absorbing material.

It is another object of the present invention to provide noel methods of protecting hair or skin by topically applying such a UV-absorbing material or a UV absorption composition to the hair or skin.

It is another object of the present invention to provide novel methods for protecting a surface from UV radiation by applying such a UV-absorbing material or a UV absorption composition to the surface.

These and other objects, which will become apparent during the following detailed description, have been achieved by the inventor's discovery that by making certain structural changes to certain known UV-absorbing materials it is possible to produce a very soluble UV-absorbing material which is adsorbed well by the skin and hair.

Thus, the present invention provides the following embodiments:
(1) An ultraviolet absorption composition, comprising at least one active ingredient selected from the group of amide-containing guanidine compounds represented by formula (I): 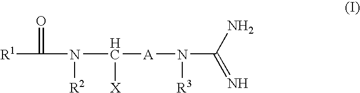
   wherein:
R¹ is a hydrocarbon group (which may contain a heteroatom on the carbon chain) which shows a maximum absorption wavelength at 260 to 360 nm when in the form of R¹COOH;
R² and R³ are each independently a hydrogen atom, a straight or branched chain alkyl group of 1 to 4 carbon atoms, or a straight or branched alkenyl group of 1 to 4 carbon atoms;
A is a straight or branched chain alkylene group of 1 to 10 carbon atoms or a straight or branched chain alkenylene group of 1 to 10 carbon atoms; and
is a hydrogen atom or carboxyl group,
   and salts thereof.
(2) An ultraviolet absorption composition, comprising at least one active ingredient selected from the group of amide-containing guanidine compounds represented by formula (II): 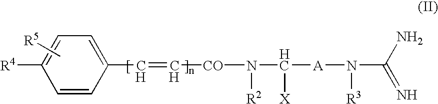
   wherein:

R² and R³ are each independently a hydrogen atom, a straight or branched chain alkyl group of 1 to 4 carbon atoms, or a straight or branched chain alkenyl group of 1 to 4 carbon atoms;
R⁴ and R⁵ are each independently a hydrogen atom, a halogen atom, a hydroxyl group, an alkyl group of 1 to 22 carbon atoms, an alkoxy group of 1 to 22 carbon atoms, an amino group, a monoalkylamino group of 1 to 22 carbon atoms, or a dialkylamino group of 1 to 22 carbon atoms;
n is 0 or 1;
A is a straight or branched chain alkylene group of 1 to 10 carbon atom or a straight or branched chain alkenylene group of 1 to 10 carbon atom; and
X is a hydrogen atom or carboxyl group,
   and salts thereof.
(3) An ultraviolet absorption composition as described in the above item (1), wherein R¹ in formula (I) is the same as R¹ in the form of R¹COOH selected from the group of cinnamic acid, p-hydroxycinnamic acid, p-alkoxycinnamic acid, 4-hydroxy-3-methoxycinnamic acid, and 3,4-dialkoxycinnamic acid.
(4) An ultraviolet absorption composition as described in any one of the above items (1) to (3), wherein in formula (I) or (II), R², R³, and X are hydrogen atoms, and A is a straight chain alkylene group of 3 carbon atoms.
(5) An ultraviolet absorption composition as described in any one of the above items (1) to (3), wherein in formula (I) or (II), R² and R³ are hydrogen atoms, X is a carboxyl group, and A is an alkylene group of 3 carbon atoms.
(6) An ultraviolet absorption hair-care product, skin-care product, or make-up cosmetic product, which comprises one or more members selected from the group of amide-containing guanidine derivatives of formula (I) or (II) and salts thereof.
(7) An amide-containing guanidine compound represented by formula (III): 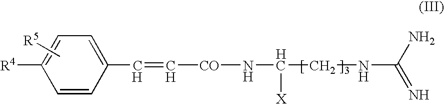
   wherein:
R⁴ and R⁵ are each independently a hydrogen atom, a hydroxyl group, an alkyl group of 1 to 22 carbon atoms, an alkoxy group of 1 to 22 carbon atoms, an amino group, a monoalkylamino group of 1 to 22 carbon atoms, or a dialkylamino group of 1 to 22 carbon atoms; and
X is a hydrogen atom or carboxyl group; and salts thereof,
provided that the following cases are excluded:
(a) a case in which R⁴ and R⁵ are hydrogen atoms, when X is a carboxyl group;
(b) a case in which R⁴ is a hydroxyl group and R⁵ is a hydrogen atom, when X is a hydrogen atom;
(c) a case in which R⁴ is a methoxyl group and R⁵ is a hydrogen atom, when X is a hydrogen atom;
(d) a case in which R⁴ is a hydroxyl group and R⁵ is a methoxyl group, when X is a hydrogen atom; and
(e) a case in which R⁴ and R⁵ are methoxyl groups, when X is a hydrogen atom.

### DETAILED DESCRIPTION OF THE PREFERRED EMBODIMENTS

The invention will be explained in details as follows.

R¹ is a hydrocarbon group, which exhibits a maximum absorption wavelength at 260 to 360 nm when in the form of R¹COOH, and which may contain a heteroatom in the carbon chain. In other words, R¹ is selected from the corresponding group in those R¹COOH compounds which exhibit a maximum absorption wavelength at 260 to 360 nm. That is, R¹COOH per se is generally described as a carboxylic acid-type UV-absorbing material which contains an aromatic ring. Specifically, it includes benzoic acids such as p-aminobenzoic acid, p-dialkylaminobenzoic acid, p-hydroxybenzoic acid, p-alkoxybenzoic acid, and the like, and cinnamic acids such as p-alkoxycinnamic acid, 4-hydroxy-3-alkoxycinnamic acid, and the like. In particular, as the starting material used in production of the present UV absorbents (UV-absorbing materials) as active ingredients in the UV absorption compositions of the present invention, a cinnamic acid represented by the above general formula (II) is preferably used in view of its easy availability and effectiveness; more preferably, cinnamic acid, p-hydroxycinnamic acid, p-alkoxycinnamic acid, 4-hydroxy-3-methoxycinnamic acid, and 3,4-dialkoxycinnamic acid are included; and particularly preferred are cinnamic acid and p-alkoxycinnamic acid. In this connection, the UV absorbent represented by the above general formula (II) includes those compounds of the above general formula (I).

The wavelength showing the maximum absorption was measured by means of a UV spectrometer in a water and/or alcohol solution of R¹COOH or its salt as mentioned in detail in the Examples.

As for the acid used in formation of a salt of the amide-containing guanidine derivative, there is no particular limitation as far as it causes no problems in the contact with the human body, particularly skin and/or hair. For example, inorganic acids (mineral acid) such as hydrochloric acid or sulfuric acid, organic acids such as citric acid, lactic acid or acetic acid, or amino acid compounds such as 2-pyrrolidone-5-carboxylic acid, glutamic acid or aspartic acid, are exemplified.

There is no particular limitation in production of the amide-containing guanidine derivatives or their salts of the present invention, and they can be produced in a conventional way. According to the method as described in JP-A-6-312972/1994, for example, a guanidyl group can be introduced into a methyl ester of a carboxylic acid-type UV absorbing material through a monoamide-amine. The desired products can also be produced by a condensation reaction of an amino-containing guanidine derivative with an acid anhydride, alkyl ester, or acid chloride of a carboxylic acid-type UV absorbent. The amino-containing guanidine derivative or its salt used in this reaction may be synthesized in a well-known method, for example, enzymatic reaction as described in Can. J. Chem., vol. 60, pp. 2810-2820 (1982), or chemical reaction as described in Z. Physiol. Chem., vol. 68, pp. 170-172 (1910).

The ultraviolet absorption compositions of the present invention which contain an amide-containing guanidine derivative or its salt as active ingredient can be prepared by adding diluents such as water and/or alcohol to the amide-containing guanidine derivative or its salt (compositions in a narrow sense); this includes the amide-containing guanidine derivative or its salt per se of the invention (compositions in a broad sense).

The amide-containing guanidine derivatives or salts thereof have an ultraviolet absorption capability and can be blended with a variety of cosmetics. The cosmetic formulation is optional and includes solution systems, solubilization systems, emulsion systems, pulverization systems, and the like. Such cosmetic formulation may be used in a hair-care product such as a shampoo, hair rinse, hair conditioner, hair cream, or hair setting preparations, a skin-care product such as a skin lotion, emulsion, cream, or facial mask, or make-up cosmetic products such as foundation cosmetics, lip sticks or eye shadow. Particularly, when used in hair, the guanidine derivatives of the invention are highly effective and show a high persistency of UV absorption effect since they have a high adsorption capability to hair and resistance to wash-out by rinsing, i.e., anti-rinsing properties. In other words, these amide-containing guanidine derivatives or salts thereof can be substituted for the known UV-absorbing materials (e.g., benzophenone derivatives, methoxycinnamic acid derivatives, and salicylic acid derivatives).

The amide-containing guanidine derivatives or salts thereof of the present invention can also be used as surface protective agents for textiles or paper from ultraviolet ray. In addition, they may be used industrially by adding as UV absorbents to water paint.

Though the amount to be blended depends on the kind of composition desired and the type of cosmetics to be blended, the guanidine derivative usually may be used in the range of 0.01 to 10 wt %, preferably 0.1 to 7 wt %, more preferably 0.5 to 7 wt %, based on the total weight of the composition, relative to the solubility and its effect.

In addition to the above-mentioned diluents, it is possible to add a variety of additives used in this field to the ultraviolet absorption compositions containing the amide-containing guanidine derivatives or salts thereof used in the present invention in such a degree that they have no adverse effect. Such additives include those conventionally used in cosmetics and dermal external preparations, for example, ionic surface active agents, non-ionic surface active agents, moisture-retaining agents, powders, pigments, oils, anti-oxidants, thickeners, film forming agents, organic solvents, preservatives, chelating agents, perfumes, and the like.

In particular, the compounds represented by the above general formula (III) are novel compounds which are expected to have performance as UV absorbents as well as to be applicable to cosmetics and other utility, though they are included in the compounds represented by the above general formula (II).

In view of easy availability of the starting materials and the degree of UV absorption capability, it is appropriate to use the compounds of the above general formula (II) wherein R⁴ is a hydrogen atom or an alkoxy group of 1 to 18 carbon atoms, R⁵ is a hydrogen atom, and X is a hydrogen atom or carboxyl group (provided that the cases wherein R⁴ and R⁵ are hydrogen atoms, and X is a carboxyl group; and R⁴ is a methoxy group and R⁵ and X are hydrogen atoms, are excluded), more preferably, the compounds wherein R⁴ is a hydrogen atom or an alkoxy group of 1 to 12 carbon atoms, R⁵ is a hydrogen atom, and X is a hydrogen atom or a carboxyl group (provided that the cases wherein R⁴ and R⁵ are hydrogen atoms, and X is a carboxyl group; and R⁴ is a methoxy group and R⁵ and X are hydrogen atoms, are excluded), and particularly the compounds wherein R⁴, R⁵ and X all are hydrogen atoms (the starting material, i.e., carboxylic acid-type UV-absorbing material, is cinnamic acid, which is bound to agmatine), wherein R⁴ is a methoxy group, R⁵ is a hydrogen atom, and X is a carboxyl group (the starting material, i.e., carboxylic acid-type UV-absorbing material, is p-methoxycinnamic acid, which is bound to arginine), and wherein R⁴ is a dodecyloxy group, R⁵ is a hydrogen atom, and X is a hydrogen atom or a carboxyl group (the raw material, i.e., carboxylic acid-type UV-absorbing material, is p-dodecyloxycinnamic acid, which is bound to arginine or agmatine).

Other features of the invention will become apparent in the course of the following descriptions of exemplary embodiments which are given for illustration of the invention and are not intended to be limiting thereof.

### EXAMPLES

### Preparation 1. Synthesis of N-cinnamoylamidobutylguanidine ½ sulfate

Synthesis of Compound 1

To a solution of 72.5 g (0.318 mol) of 4-aminobutylguanidine sulfate in 425 g of water was added 200 g of isopropyl alcohol (IPA), and the mixture was adjusted to pH 11 with 27% NaoH. There were added 51.3 g (0.308 mol) of cinnamoyl chloride and 27% NaOH while the mixture was kept at a temperature of 15° C. and pH 11. After addition of cinnamoyl chloride, the mixture was stirred at the same temperature and pH for 30 minutes. The mixture was then warmed up to 40° C. to separate into 2 layers. To the upper layer was added 300 g of water, and about 300 g of water/IPA was then removed by distillation under reduced pressure. The condensed liquid after addition of 250 g of water and cooling yielded crystals as a precipitate. The precipitate was washed with water to give 67.4 g of N-cinnamoylamidobutylguanidine ½ sulfate as crude crystals. These crude crystals were recrystallized from a mixture of IPA and water to give 60.7 g (0.196 mol; yield: 63.6%) of N-cinnamoylamidobutylguanidine ½ sulfate as white powder. The resulting compound was analyzed to give the following data.
¹H-NMR (CD₃OD) δ: 1.65 (br. 4H), 3.21 (m, 2H), 3.34 (m, 2H), 6.65 (d, 1H), 7.38 (m, 3H), 7.50 (d, 1H), 7.55 (m, 2H).

¹³C-NMR (DMSO-d6) δ: 157.39 (guanidine), 165.56 (amide)

ESI-Mass: 261 (MH+)

### Preparation 2. Synthesis of N-cinnamoylamidobutylguanidine hydrochloride

Synthesis of Compound 2

In a mixture of 13 g of IPA and 12 g of water was dissolved 3.0 g (9.7 mmol) of N-cinnamoylamidobutylguanidine ½ sulfate prepared in Preparation 1. There was added a solution of 1.2 g (4.9 mmol) of barium chloride dihydrate in 15 ml of water. The precipitate was filtered off, and the filtrate concentrated under reduced pressure to give 2.6 g (8.8 mmol) of N-cinnamoylamidobutylguanidine hydrochloride.

### Preparation 3. Synthesis of N-paramethoxycinnamoylarginine

Synthesis of Compound 3

To a suspension of 25.6 g (147.0 mmol) of arginine in 66 g of water and 153 g of t-BuOH was added a solution of 27.5 g (140.0 mmol) of paramethoxycinnamoyl chloride in toluene and 27% NaOH at 20° C. and pH 10. After addition of paramethoxycinnamoyl chloride, the mixture was stirred at the same temperature and pH for 30 minutes. Then the reaction mixture was adjusted to pH 5 with addition of concentrated hydrochloric acid to give crystals as precipitate. The latter was washed with water and ethanol to give 15.3 g of crude crystals, which were recrystallized from a mixture of IPA and water to give 13.0 g (38.9 mmol; yield: 27.8%) of pure N-paramethoxycinnamoylarginine as white powder. The resulting compound were analyzed to give the following data.

¹H-NMR (CD₃OD) δ: 1.66 (m, 2H), 1.77 (m, 1H), 1.93 (m, 1H), 3.22 (m, 2H), 3.81 (s, 3H), 4.42 (t, 1H), 6.57 (d, 1H), 6.92 (m, 2H), 7.44 (d, 1H), 7.49 (m, 2H).

¹³C-NMR (DMSO-d6) δ: 157.59 (guanidine), 164.87 (amide), 176.67 (carbonyl)

ESI-Mass: 335 (MH+)

### Preparation 4. Synthesis of N-paradodecyloxycinnamoylamidobutylguanidine ½ sulfate

Synthesis of Compound 4

A solution of 11.5 g (50.4 mmol) of 4-aminobutylguanidine sulfate dissolved in 100 g of water and 95 g of t-BuOH was adjusted to pH 11 with 27% NaOH. A solution of 15.8 g (45.1 mmol) of paradodecyloxycinnamoyl chloride dissolved in toluene and 27% NaOH were added thereto to maintain a pH of 11 at 25° C. After addition of paradodecyloxycinnamoyl chloride, the mixture was stirred at the same temperature and pH for 30 minutes. The reaction mixture was then adjusted to pH 3 with addition of 75% sulfuric acid to give crystals as a precipitate. The latter was washed with water, ethanol and chloroform, and then recrystallized from a mixture of water and t-BuOH to give 6.7 g (13.6 mmol; yield: 30.1%) of pure N-paradodecyloxycinnamoylamidobutylguanidine ½ sulfate as white powder. The resulting compound was analyzed to give the following data.

¹H-NMR (CD₃OD) δ: 0.89 (t, 3H), 1.29 (br, 16H), 1.47 (m, 2H), 1.64 (br, 4H), 1.77 (m, 2H), 3.20 (m, 2H), 3.33 (m, 2H), 3.99 (t, 2H), 6.51 (d, 1H), 6.91 (m, 2H), 7.46 (d, 1H), 7.50 (m, 2H).

ESI-Mass: 445 (MH+)

### Preparation 5. Synthesis of N-paradodecyloxycinnamoylamidobutylguanidine hydrochloride

Synthesis of Compound 5

In the same manner as in Preparation 2, the reaction was conducted using 3.0 g (6.1 mmol) of N-paradodecyloxycinnamoylamidobutylguanidine ½ sulfate of Preparation 4 in place of 3.0 g (9.7 mmol) of N-cinnamoylamidobutylguanidine ½ sulfate and using a mixture of 36 g of t-BuOH and 38 g of water in place of a mixture of 13 g of IPA and 12 g of water to give 2.5 g (5.2 mmol) of N-paradodecyloxycinnamoylamidobutylguanidine hydrochloride.

### Preparation 6. Synthesis of N-paradodecyloxycinnamoylarginine

Synthesis of Compound 6

To a suspension of 8.3 g (47.6 mmol) of arginine in 80 g of water and 103 g of t-BuOH was added a solution of 15.0 g (42.8 mmol) of paradodecyloxycinnamoyl chloride in toluene and 27% NaOH at 25° C. and pH 10. After addition of paradodecyloxycinnamoyl chloride, the mixture was stirred at the same temperature and pH for 30 minutes. Then the reaction mixture was adjusted to pH 3 with addition of concentrated sulfuric acid to give crystals as a precipitate. The latter was washed with water and t-BuOH, and then recrystallized from a mixture of water and t-BuOH to give 7.3 g (15.0 mmol; yield: 35.0%) of pure N-paradodecyloxycinnamoylarginine as white powder. The resulting compound was analyzed to give the following data.

¹H-NMR (CD₃OD) δ: 0.89 (t, 3H), 1.29 (br, 16H), 1.47 (m, 2H), 1.65 (m, 2H), 1.78 (m, 3H), 1.93 (m, 1H), 3.22 (m, 2H), 4.00 (t, 2H), 4.42 (t, 1H), 6.58 (d, 1H), 6.91 (m, 2H), 1.78 (m, 7.46 (d, 1H), 7.49 (m, 2H).

ESI-Mass: 489 (MH+)

Test 1. Wavelength at the Maximum Absorption

Table 1 shows the wavelength at the UV absorption maximum and the molar absorption coefficients measured for the amide-containing guanidine derivatives obtained in Preparations and the starting carboxylic acid UV absorbents in water/alcohol solutions (30% methanol solution for Preparations 1 and 3; 40% isopropyl alcohol solution for Preparations 4 and 6).

TABLE 1

| | Amide-containing guanidine | | | |
|---|---|---|---|---|
| | derivative | | Starting UV absorbent | |
| | Wavelength | Mol.Abs.Coef. | Wavelength | Mol.Abs.Coef. |
| | (nm) | (ε) | (nm) | (ε) |
| Prep.1 | 280 | 21885 | 279 | 18661 |
| Prep.3 | 307 | 24977 | 307 | 20845 |
| Prep.4 | 307 | 24122 | 309 | 21246 |
| Prep.6 | 308 | 25740 | as above | as above |

As shown in Table 1, it is found that the absorption wavelength and the molar absorption coefficient of the amide-containing guanidine derivative are approximately the same as those of the starting UV absorbent carboxylic acid.

Test 2. Solubility

Table 2 shows the solubility of the amide-containing guanidine derivatives and of the starting carboxylic acid-type UV absorbents in water at room temperature.

TABLE 2

| | Amide-containing | Starting UV |
|---|---|---|
| | guanidine derivative (wt %) | absorbent (wt %) |
| Compound 2 | >15 | <0.5 |
| Compound 3 | >15 | <0.1 |
| (HCl salt) | | |
| Compound 5 | >7 | <0.01 |

As shown in Table 2, it is found that the conversion into the guanidine derivatives increases the solubility in water.

Test 3. Adsorption on Hair

The adsorptive properties of Compound 1 as mentioned above as the amide-containing guanidine derivative or its salt of the present invention and of cinnamidopropyl trimethylammonium chloride (INCROQUAT UV-283/CRODA) as a reference material were investigated on hair.

Experimental method: To a solution of 5 mg of Compound 1 in 30 ml of water was added 0.3 g of hair (5 mm in length) pretreated by bleaching, and the mixture was stirred at 40° C. for 30 minutes. Before and after addition of the hair, the amount of Compound 1 in the aqueous solution was determined by HPLC, and the loss in the solution was regarded as the amount adsorbed on the hair.

Result: Compound 1 was adsorbed on hair at a proportion of about 3.5 mmol per 100 g of hair. In contrast, the reference material was adsorbed at a proportion of about 1.7 mmoly.

Blend 1.

Using Compound 2 prepared in Preparation 2, a hair rinse product (hair care cosmetic) was prepared according to the prescription as shown in the following Table 3.

TABLE 3

| Component A | |
|---|---|
| Cetostearyl alcohol | 3.0 wt % |
| Octyldodecyl myristate | 1.0 |
| Glyceryl monostearate | 1.0 |
| Component B | |
| Monostearyltrimethylammonium chloride | 2.0 |
| (50%) | |
| Compound 2 | 2.0 |
| 1,3-Butylene glycol | 3.0 |
| Antiseptic agent | adequate amt. |
| Purified water | residual |
| Total | 100.0 |

That is, Component A and Component B were respectively dissolved at 80° C., and Component A was added to Component B to form an emulsion. This was then cooled to 30° C. to give a product.

The resulting hair rinse product gives an excellent feeling, and after drying it gives a wet feel and care of hair becomes easy.

Blend 2.

Using Compound 5 prepared in Preparation 5, a hair rinse product (hair care cosmetic) was prepared according to the prescription as shown in the following Table 4.

TABLE 4

| Component A | |
|---|---|
| Cetostearyl alcohol | 3.0 wt % |
| Octyldodecyl myristate | 1.0 |
| Glyceryl monostearate | 1.0 |
| Component B | |
| Monostearyltrimethylammonium chloride | 2.0 |
| (50%) | |
| Compound 5 | 1.0 |
| 1,3-Butylene glycol | 3.0 |
| Antiseptic agent | adequate amt. |
| Purified water | residual |
| Total | 100.0 |

That is, Component A and Component B were respectively dissolved at 80° C., and Component A was added to Component B to form an emulsion. This was then cooled to 30° C. to give a product.

The resulting hair rinse product gives an excellent feeling, and after drying it gives a light finishing feel and care of hair becomes easy.

Blend 3.

Using Compound 2 prepared in Preparation 2, a skin lotion (skin care cosmetic) was prepared according to the prescription as shown in the following Table 5.

TABLE 5

| Sodium DL-pyrrolidonecarboxylate (50%) | 3.0 wt % |
|---|---|
| Sorbitol (70%) | 3.0 |
| Compound 2 | 7.0 |
| Carboxymethylcellulose | 0.3 |
| Antiseptic agent | adequate amt. |
| Purified water | residual |
| Total | 100.0 |

That is, each component was mixed and dissolved at 70-80° C. with stirring. The mixture was cooled to 30° C. with stirring to give a product.

The resulting skin lotion gave a good feeling without stickiness.

Blend 4.

Using Compound 3 prepared in Preparation 3, a hair gel product (hair care cosmetic) was prepared according to the prescription as shown in the following Table 6.

TABLE 6

| Carboxyvinylpolymer | 0.7 wt % |
|---|---|
| Polyvinylpyrrolidone | 2.0 |
| Glycerin | 2.0 |
| Ethanol | 25.0 |
| Sodium hydroxide | adequate amt. |
| Compound 3 | 3.0 |
| Polyoxyethylene octyl dodecyl ether | 0.3 |
| Antiseptic agent | adequate amt. |
| Purified water | residual |
| Total | 100.0 |

That is, carboxyvinylpolymer was dispersed in glycerin and a part of purified water, to which was added the other components with stirring to give a product.

The resulting hair gel product was rich in setting potency and gave a good feeling without stickiness.

Blend 5.

Using Compound 6 prepared in Preparation 6, a hair shampoo product (hair care cosmetic) was prepared according to the prescription as shown in the following Table 7.

TABLE 7

| Coconut oil fatty acid acylglutamic acid | 10.0 wt % |
|---|---|
| triethanolamine solution (30%) | |
| Polyoxyethylene lauryl ether | 26.0 |
| sodium sulfate solution (27%) | |
| Coconut oil fatty acid diethanolamide | 5.0 |
| Propylene glycol | 5.0 |
| Compound 6 | 1.5 |
| Cationic cellulose | 0.3 |
| Antiseptic agent | adequate amt. |
| Purified water | residual |
| Total | 100.0 |

That is, each component was mixed and dissolved at 70-80° C. with stirring. The mixture was cooled to 30° C. with stirring to give a product.

Using the resulting hair shampoo product, care of hair after drying becomes easy.

### INDUSTRIAL APPLICABILITY

The amide-containing guanidine derivatives or salts thereof of the preent invention have a UV absorption power, are soluble in water and readily adsorbed onto skin or hair, and resistant to rinsing. Accordingly, they can be blended with hair-care cosmetic products, particularly, of wash-out type, such as shampoo or hair rinse. Further, they can be blended with skin care cosmetic products, particularly, skin lotion, since they are highly soluble in water.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An ultraviolet absorption composition, comprising at least one active ingredient selected from the group of amide-containing guanidine compounds represented by formula (I): 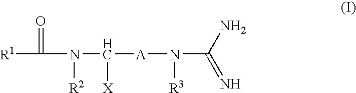
wherein:
R¹ is a hydrocarbon group, which may contain a heteroatom in the carbon chain, and which exhibits a maximum absorption wavelength at 260 to 360 nm when in the form of R¹COOH;
R² and R³ are each independently a hydrogen atom, a straight or branched chain alkyl group of 1 to 4 carbon atoms, or a straight or branched chain alkenyl group of 1 to 4 carbon atoms;
A is a straight or branched chain alkylene group of 1 to 10 carbon atoms or a straight or branched chain alkenylene group of 1 to 10 carbon atoms; and
X is a hydrogen atom or carboxyl group,
and salts thereof, and mixtures thereof.

2. An ultraviolet absorption composition as claimed in claim 1, wherein R¹ in formula (I) is the same as R¹ in the formula R¹COOH, and R¹COOH is selected from the group consisting of cinnamic acid, p-hydroxycinnamic acid, p-alkoxycinnamic acid, 4-hydroxy-3-methoxycinnamic acid, and 3,4-dialkoxycinnamic acid.

3. An ultraviolet absorption composition as claimed in claim 2, wherein in formula (I), R², R³ and X are hydrogen atoms, and A is a straight chain alkylene group of 3 carbon atoms.

4. An ultraviolet absorption composition as claimed in claim 2, wherein in formula (I), R² and R³ are hydrogen atoms, X is a carboxyl group, and A is an alkylene group of 3 carbon atoms.

5. An ultraviolet absorption composition as claimed in claim 1, wherein in formula (I), R², R³ and X are hydrogen atoms, and A is a straight chain alkylene group of 3 carbon atoms.

6. An ultraviolet absorption composition as claimed in claim 1, wherein in formula (I), R² and R³ are hydrogen atoms, X is a carboxyl group, and A is an alkylene group of 3 carbon atoms.

7. An ultraviolet absorption, composition comprising at least one active ingredient selected from the group of amide-containing guanidine compounds represented by formula (II): 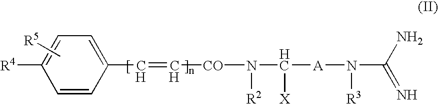
wherein:
R² and R³ are each independently a hydrogen atom, a straight or branched chain alkyl group of 1 to 4 carbon atoms or a straight or branched chain alkenyl group of 1 to 4 carbon atoms;
R⁴ and R⁵ are each independently a hydrogen atom, a halogen atom, a hydroxyl group, an alkyl group of 1 to 22 carbon atoms, an alkoxy group of 1 to 22 carbon atoms, an amino group, a monoalkylamino group of 1 to 22 carbon atoms, or a dialkylamino group of 1 to 22 carbon atoms; n is 0 or 1;
A is a straight or branched chain alkylene group of 1 to 10 carbon atom or a straight or branched chain alkenylene group of 1 to 10 carbon atom; and
X is a hydrogen atom or carboxyl group,
and salts thereof, and mixtures thereof.

8. An ultraviolet absorption composition as claimed in claim 7, wherein in formula (II), R², R³ and X are hydrogen atoms, and A is a straight chain alkylene group of 3 carbon atoms.

9. An ultraviolet absorption composition as claimed in claim 7, wherein in formula (II) R² and R³ are hydrogen atoms, X is a carboxyl group, and A is an alkylene group of 3 carbon atoms.

10. An ultraviolet absorption composition as claimed in claim 1, which is in the form of a hair-care product, skin-care product, or a make-up cosmetic product.

11. An ultraviolet absorption composition as claimed in claim 1, which is in the form of a surface protective agent for textiles or paper.

12. An ultraviolet absorption composition as claimed in claim 7, which is in the form of a hair-care product, skin-care product, or a make-up cosmetic product.

13. An amide-containing guanidine compound represented by formula (III): 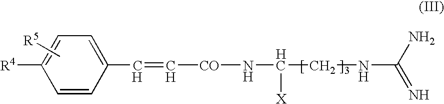
wherein:
R⁴ and R⁵ are each independently a hydrogen atom, a hydroxyl group, an alkyl group of 1 to 22 carbon atoms, an alkoxy group of 1 to 22 carbon atoms, an amino group, a monoalkylamino group of 1 to 22 carbon atoms, or a dialkylamino group of 1 to 22 carbon atoms; and
X is a hydrogen atom or carboxyl group;
or a salt thereof,
provided that the following cases are excluded:
(a) a case in which R⁴ and R⁵ are hydrogen atoms, when X is a carboxyl group;
(b) a case in which R⁴ is a hydroxyl group and R⁵ is a hydrogen atom, when X is a hydrogen atom;
(c) a case in which R⁴ is a methoxyl group and R⁵ is a hydrogen atom, when X is a hydrogen atom;
(d) a case in which R⁴ is a hydroxyl group and R⁵ is a methoxyl group, when X is a hydrogen atom; and
(e) a case in which R⁴ and R⁵ are methoxyl groups, when X is a hydrogen atom.

14. A method of protecting skin or hair from UV radiation, comprising topically applying to skin or hair an effective amount of at least one amide-containing guanidine compound represented by formula (I): 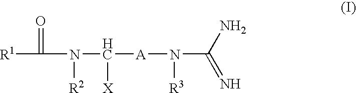
wherein:
R¹ is a hydrocarbon group, which may contain a heteroatom in the carbon chain, and which exhibits a maximum absorption wavelength at 260 to 360 nm when in the form of R¹COOH;
R² and R³ are each independently a hydrogen atom, a straight or branched chain alkyl group of 1 to 4 carbon atoms, or a straight or branched chain alkenyl group of 1 to 4 carbon atoms;
A is a straight or branched chain alkylene group of 1 to 10 carbon atoms or a straight or branched chain alkenylene group of 1 to 10 carbon atoms; and
X is a hydrogen atom or carboxyl group,
or a salts thereof.

15. The method of claim 14, wherein R¹ in formula (I) is the same as R¹ in the formula R¹COOH, and R¹COOH is selected from the group consisting of cinnamic acid, p-hydroxycinnamic acid, p-alkoxycinnamic acid, 4-hydroxy-3-methoxycinnamic acid, and 3,4-dialkoxycinnamic acid.

16. The method of claim 15, wherein in formula (I), R², R³ and X are hydrogen atoms, and A is a straight chain alkylene group of 3 carbon atoms.

17. The method of claim 15, wherein in formula (I), R² and R³ are hydrogen atoms, X is a carboxyl group, and A is an alkylene group of 3 carbon atoms.

18. A method of protecting skin or hair from UV radiation, comprising topically applying to skin or hair an effective amount of at least one amide-containing guanidine compound represented by formula (II): 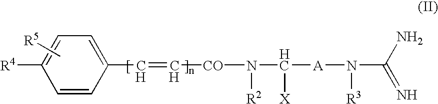
wherein:
R² and R³ are each independently a hydrogen atom, a straight or branched chain alkyl group of 1 to 4 carbon atoms or a straight or branched chain alkenyl group of 1 to 4 carbon atoms;
R⁴ and R⁵ are each independently a hydrogen atom, a halogen atom, a hydroxyl group, an alkyl group of 1 to 22 carbon atoms, an alkoxy group of 1 to 22 carbon atoms, an amino group, a monoalkylamino group of 1 to 22 carbon atoms, or a dialkylamino group of 1 to 22 carbon atoms; n is 0 or 1;
A is a straight or branched chain alkylene group of 1 to 10 carbon atom or a straight or branched chain alkenylene group of 1 to 10 carbon atom; and
X is a hydrogen atom or carboxyl group,
or a salt thereof.

19. The method of claim 18, wherein in formula (II), R², R³ and X are hydrogen atoms, and A is a straight chain alkylene group of 3 carbon atoms.

20. The method of claim 18, wherein in formula (II) R² and R³ are hydrogen atoms, X is a carboxyl group, and A is an alkylene group of 3 carbon atoms.
